(19) United States
(12) Patent Application Publication
(10) Pub. No.: US 2009/0270964 A1
(43) Pub. Date: Oct. 29, 2009

(54) Toroidal balloon system and method of use

(75) Inventors: Meredith Huetter, Santa Rosa, CA (US); Theodore Morris, Santa Rosa, CA (US); Ryan Ferrasci, Huntington Beach, CA (US); Susan Rea, Santa Rosa, CA (US); Rany Huynh, Charlestown, MA (US)

Correspondence Address: MEDTRONIC VASCULAR, INC.;IP LEGAL DEPARTMENT, 3576 UNOCAL PLACE, SANTA ROSA, CA, 95403, US

(73) Assignee: Medtronic Vascular, Inc., Santa Rosa, CA (US)

(21) Appl. No.: 12/108,725

(22) Filed: Apr. 24, 2008

(51) Int. Cl. A61F 2/06; A61M 25/10; A61M 29/02

(52) U.S. Cl. 623/1.11; 604/96.01; 604/103; 29/428; 623/1.23; 128/898; 606/200

(57) ABSTRACT

A toroidal balloon system apparatus and method for use in a vessel includes a catheter defining an inflation lumen and having an inflation port in communication with the inflation lumen; and a toroidal balloon attached to the catheter. The toroidal balloon defines a balloon lumen in communication with the inflation port to inflate the balloon and a central lumen which allows fluid flow through the balloon and the vessel when the toroidal balloon is inflated.

### TECHNICAL FIELD

The technical field of this disclosure is medical implantation devices, particularly, a toroidal balloon system and method of use.

### BACKGROUND OF THE INVENTION

Wide ranges of medical treatments have been developed using endoluminal prostheses, which are medical devices adapted for temporary or permanent implantation within a body lumen, such as naturally occurring or artificially made lumens. Examples of lumens in which endoluminal prostheses may be implanted include arteries such as those located within coronary, mesentery, peripheral, or cerebral vasculature; arteries; gastrointestinal tract; biliary tract; urethra; trachea; hepatic shunts; and fallopian tubes. Various types of endoluminal prostheses have also been developed with particular structures to modify the mechanics of the targeted lumen wall.

A number of vascular devices have been developed for replacing, supplementing, or excluding portions of blood vessels. These vascular devices include endoluminal vascular prostheses and stent grafts. Aneurysm exclusion devices, such as abdominal aortic aneurysm (AAA) devices, are used to exclude vascular aneurysms and provide a prosthetic lumen for the flow of blood. Vascular aneurysms are the result of abnormal dilation of a blood vessel, usually from disease or a genetic predisposition, which can weaken the arterial wall and allow it to expand. Aneurysms can occur in any blood vessel, but most occur in the aorta and peripheral arteries, with the majority of aneurysms occurring in the abdominal aorta. An abdominal aneurysm typically begins below the renal arteries and may extend into one or both of the iliac arteries.

Aneurysms, especially abdominal aortic aneurysms, have been commonly treated in open surgery procedures where the diseased vessel segment is bypassed and repaired with an artificial vascular graft. While open surgery is an effective surgical technique in light of the risk of a fatal abdominal aortic aneurysm rupture, the open surgical technique suffers from a number of disadvantages. It is complex, requires a long hospital stay, requires a long recovery time, and has a high mortality rate. Less invasive devices and techniques have been developed to avoid these disadvantages. Tubular endoluminal prostheses that provide a lumen or lumens for blood flow while excluding blood flow to the aneurysm site are introduced into the blood vessel using a catheter in a less or minimally invasive technique. The tubular endoluminal prosthesis is introduced in a small diameter compressed configuration and expanded at the aneurysm. Although often referred to as stent grafts, these tubular endoluminal prostheses differ from so called covered stents in that they are not used to mechanically prop open stenosed natural blood vessels. Rather, they are used to secure graft material in a sealing engagement with the vessel wall and to prop open the tubular passage through the graft without further opening the abnormally dilated natural blood vessel.

Stent grafts for use in abdominal aortic aneurysms typically include a support structure supporting woven or interlocked graft material. Examples of woven graft materials are woven polymer materials, e.g., Dacron, or polytetrafluoroethylene (PTFE). Interlocked graft materials include knit, stretch, and velour materials. The graft material is secured to the inner or outer diameter of the support structure, which supports the graft material and/or holds it in place against a vessel wall. The stent graft is secured to a vessel wall above and below the aneurysm. A proximal spring stent of the stent graft can be located above the aneurysm to provide a radial force to engage the vessel wall and seal the stent graft to the vessel wall.

One problem is that stent grafts can migrate over time after installation in the vessel. The stent graft is subject to a variety of loads due to the force associated with blood flowing through the stent graft, and the pulsatile pressure causing expansion and contraction of arteries. Changes in the anatomy of the abdominal aortic aneurysm can also contribute to the cause of migration.

One attempt to prevent migration has been to mold the stent graft during deployment. A catheter balloon is inserted at the fixation point and inflated to shape the support structure of the stent graft. Unfortunately, the inflated catheter balloon occludes the vessel, limiting the time the clinician can perform the molding because the blood flow is blocked, which can cause complications such as ischemia if continued for significant periods of time. The quality of the molding is limited by the available occlusion time.

Another problem is that deployment of stent grafts can dislodge emboli, which can block vessels downstream of the stent graft deployment site and cause tissue damage. One attempt to avoid emboli migration has been to block the vessel downstream of the deployment site with a catheter balloon then remove any emboli between the deployment site and the balloon before deflating the catheter balloon. Unfortunately, this blocking of the blood flow can cause complications such as ischemia if continued for significant periods of time.

Yet another problem in stent graft placement is that some desirable deployment sites are inaccessible due to their small diameter and tortuous approach. Different folding and packing strategies have reduced the delivery diameter of stent grafts, but the support structure limits the diameter that can be achieved, which in turn limits the accessible deployment sites.

Yet another problem in stent graft research is the difficulty in creating aneurysms in animal models for the testing of stent grafts and other aneurysm related devices and procedures. Unsatisfactory attempts to create aneurysms have included installation of artificial fabric patches in the vessel, attack on the vessel with enzymes, and genetically modified animals. Aneurysmal devices are often tested on normal vessels without aneurysms due to the lack of good animal models.

It would be desirable to overcome the above disadvantages.

### SUMMARY OF THE INVENTION

One aspect according to the present invention provides a toroidal balloon system for use in a vessel including a catheter defining an inflation lumen and having an inflation port in communication with the inflation lumen; and a toroidal balloon attached to the catheter, the toroidal balloon defining a balloon lumen in communication with the inflation port and a central lumen for fluid flow through the vessel when the toroidal balloon is inflated.

Another aspect according to the present invention provides a method of manufacturing a toroidal balloon system including providing a toroidal balloon blank having a balloon body defining a balloon lumen, a first leg attached to the balloon body, and a second leg attached to the balloon body opposite the first leg; folding the first leg through the balloon lumen and into the second leg; folding the second leg into the balloon lumen about the first leg; providing a catheter defining a catheter lumen and having an catheter inflation port; aligning the catheter inflation port so the catheter lumen communicates with the balloon lumen; and sealing the catheter to the toroidal balloon blank.

Another aspect according to the present invention provides a method of molding a stent graft to a vessel including deploying the stent graft in the vessel, the stent graft having at least one stent; placing a toroidal balloon within the stent graft at the stent, the toroidal balloon having a central lumen; and inflating the toroidal balloon to fit the stent to the vessel while maintaining blood flow in the vessel through the central lumen.

Another aspect according to the present invention provides a method of deploying a graft in a vessel including providing a graft having a graft portion stained with Rose Bengal; isolating a vessel wall portion from blood flow through the vessel without blocking the blood flow through the vessel; staining the vessel wall portion with Rose Bengal; placing the stained graft portion adjacent the stained vessel wall portion; and exposing the stained graft portion and the stained vessel wall portion with light energy through the toroidal balloon to bond the stained graft portion and the stained vessel wall portion.

Another aspect according to the present invention provides a method of developing an aneurysm in a vessel including advancing a toroidal balloon having a central lumen to a target site in the vessel; inflating the toroidal balloon to a diameter greater than an initial vessel diameter at the target site; maintaining blood flow in the vessel through the central lumen; and retaining the toroidal balloon in the vessel until the vessel diameter at the target site is fixed at the greater diameter of the toroidal balloon.

Another aspect according to the present invention provides a system for graft deployment in a vessel including a graft having a stained graft portion; a double toroidal balloon system, and a light delivery balloon system. The double toroidal balloon system includes a first catheter defining a supply lumen and a return lumen; and a double balloon attached to the first catheter, the double balloon having a first balloon connected to a second balloon with a perfusion body, the first balloon having a first central lumen, the second balloon having a second central lumen, the perfusion body having a perfusion opening connecting the first central lumen and the second central lumen. The supply lumen and the return lumen communicate to outside the perfusion body. The light delivery balloon system includes a second catheter defining a light catheter lumen; and a toroidal balloon attached to the second catheter. The double toroidal balloon system is operable to isolate a vessel wall portion of the vessel and deliver stain to the vessel wall portion through the supply lumen to generate a stained wall portion; the light delivery balloon system is operable to align the stained graft portion with the stained wall portion; and the light delivery balloon system is further operable to deliver a light catheter through the light catheter lumen to expose the aligned stained graft portion and the stained wall portion with light.

The foregoing and other features and advantages will become further apparent from the following detailed description, read in conjunction with the accompanying drawings. The detailed description and drawings are merely illustrative.

### BRIEF DESCRIPTION OF THE DRAWINGS

FIGS. 1A-1C are schematic side, cross section, and end views, respectively, of a toroidal balloon system;

FIG. 2 is a schematic perspective view of the toroidal balloon system of FIGS. 1A-1C with an embolic net;

FIG. 3 is a schematic cross section view of another embodiment of a toroidal balloon system;

FIGS. 4A & 4B are schematic side and end views, respectively, of another embodiment of a toroidal balloon system;

FIGS. 5A-5D are schematic progressive cross sectional views of a method of manufacturing a toroidal balloon system;

FIGS. 6A-6D are progressive cross sectional views of another method of manufacturing a toroidal balloon system;

FIGS. 7A-7D are schematic progressive cross sectional views of another method of manufacturing a toroidal balloon system;

FIG. 8 is a flowchart of the steps of a method of manufacturing a toroidal balloon system;

FIG. 9 is a schematic side view of a graft;

FIGS. 10A-10C are schematic side, cross section, and end views, respectively, of a double toroidal balloon system;

FIGS. 11A & 11B are schematic side and end views, respectively, of a light delivery balloon system;

FIG. 12 is a schematic side view of a double toroidal balloon system deployed in a vessel;

FIG. 13 is a schematic side view of a light delivery balloon system deployed in a vessel;

FIG. 14 is a flowchart of the steps of a method of deploying a graft in a vessel;

FIGS. 15A-15C are schematic progressive side views of developing an aneurysm with a toroidal balloon system;

FIG. 16 is a flowchart of the steps of a method of developing an aneurysm in a vessel;

FIG. 17 is a schematic side view of a toroidal balloon system deployed in a vessel with a stent graft; and

FIG. 18 is a flowchart of the steps of a method of molding a stent graft to a vessel.

### DETAILED DESCRIPTION

Embodiments according to the invention will now be described by reference to the figures wherein like numbers refer to like structures. The terms “distal” and “proximal” are used herein with reference to the treating clinician during the use of the catheter system: “distal” indicates a delivery system portion distant from, or a direction away from the clinician and “proximal” indicates a delivery system portion near to, or a direction towards the clinician.

Stent graft devices and methods for fixation of stent grafts are disclosed. While these devices and methods are described below in terms of being used in conjunction with abdominal aortic aneurysms and thoracic aortic aneurysms, those skilled in the art will appreciate that the devices could be used in other vessels as well.

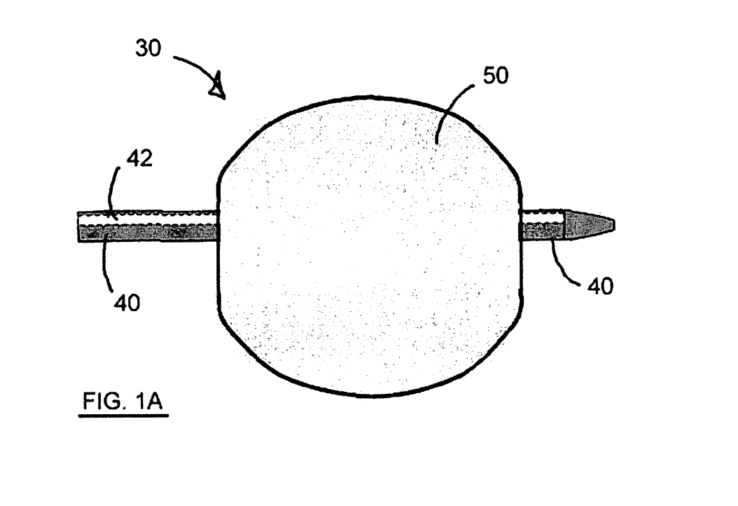

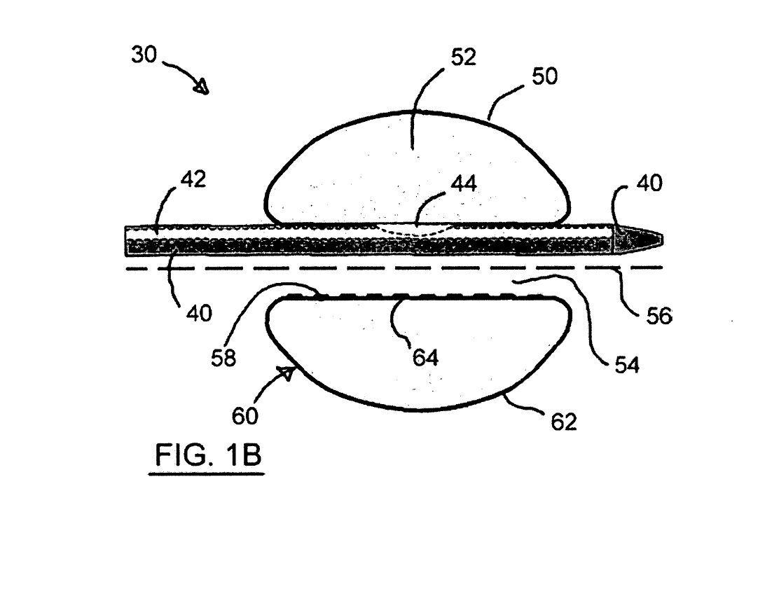

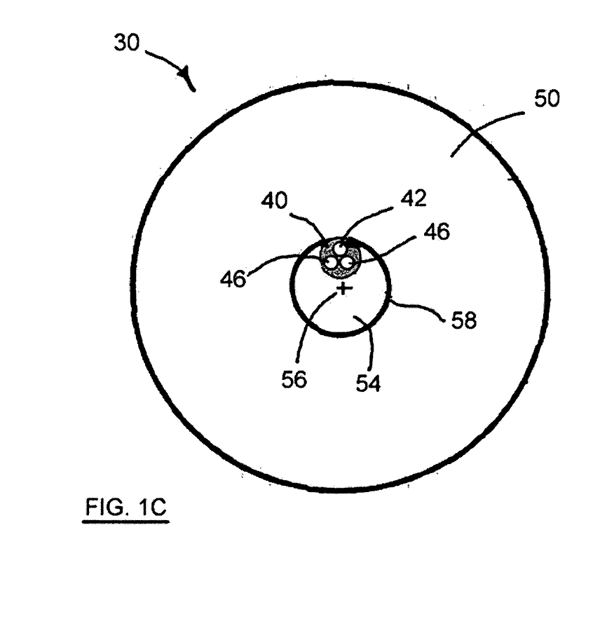

FIGS. 1A-1C are schematic side, cross section, and end views, respectively, of a toroidal balloon system. The toroidal balloon system is shown with the toroidal balloon inflated. The toroidal balloon system 30 includes a catheter 40 and a toroidal balloon 50 attached to the catheter 40. The catheter 40 defines an inflation lumen 42 and has an inflation port 44 in communication with the inflation lumen 42. The inflation port 44 can be skived into the inflation lumen 42 of the catheter 40. The toroidal balloon 50 defines a balloon lumen (interior volume) 52 in communication with the inflation port 44. When the toroidal balloon 50 is inflated, the toroidal balloon 50 defines a central lumen 54 for fluid flow through the vessel in which the toroidal balloon 50 is deployed. The toroidal balloon 50 has a balloon axis 56 through the central lumen 54. The “toroidal balloon” as shown herein is expressly defined as a balloon having a surface obtained by rotating a planar closed curve about an axis parallel to the plane which does not intersect the closed curve, wherein the portion of the closed curve away from the axis is generally semicircular and the portion of the closed curve near the axis is in a family of smooth curves between a line and a semicircle. In the embodiment illustrated in FIG. 1B, the planar closed curve 60 is generally D-shaped, with the portion 62 of the closed curve 60 away from the balloon axis 56 being generally semicircular and the portion 64 of the closed curve 60 near the balloon axis 56 being generally linear.

Referring to FIG. 1C, the catheter 40 can further define additional auxiliary lumens 46 in addition to the inflation lumen 42. The auxiliary lumens 46 can be used for the passage of guidewires, microcatheters, fiberoptic cables, other tools, and fluids, such as drugs or therapeutic agents.

The catheter 40 can be made of any flexible biocompatible material normally used for catheters. For example, the catheter 40 can be made of polymers such as polyurethane, polyethylene, polyether block amide (PEBAX), nylon, composites, or any combination of the above, or the like. The catheter 40 is long enough to reach from the clinician to the site in the vessel where the toroidal balloon 50 is to be used. The approach to the site in the vessel depends on the location of the site in the vasculature. For example, when the toroidal balloon system 30 is used in conjunction with a stent graft in an aortic aneurysm, the approach can be from the femoral artery or the carotid artery.

The toroidal balloon 50 can be made of any flexible biocompatible material normally used for catheter balloons. For example, the toroidal balloon 50 can be made of polymers such as polyethylene, polyethylene terephalate (PET), nylon, polyurethane, polyether block amide (PEBAX), polyetheretherketone (PEEK), or the like. The toroidal balloon 50 can be of uniform thickness, or can be thicker or thinner in certain portions for variable compliance. The material can be selected to make the toroidal balloon 50 more or less compliant as desired for a particular use, i.e., non-compliant, semi-compliant, or compliant. For example, a toroidal balloon 50 for molding a stent graft support structure will be less compliant to allow the toroidal balloon 50 to shape the support structure. A toroidal balloon 50 for sealing against a vessel wall will be more compliant to form a good seal and avoid damage to the vessel wall. A non-compliant toroidal balloon 50 can be used with less delicate structures, such as aneurysms, and a compliant toroidal balloon 50 can be used with more delicate structures, such as dissections. The toroidal balloon 50 can be inflated with a contrast saline solution or other liquid.

The inner wall 58 can be made of the same materials as the toroidal balloon 50 or can be made of other materials. In one embodiment, the inner wall 58 can include an internal sheath as additional support and reinforcement for the inner wall 58. The internal sheath can be a common biaxial braid or other braided pattern, which expands in diameter when axially compressed and shrinks in diameter when axially tensioned. This allows the braided internal sheath to expand in diameter when the toroidal balloon 50 is pushed out from the delivery sheath and shrink in diameter when it is pulled into the delivery sheath. The internal sheath can be made of polymers such as polyurethane, polyethylene, polyether block amide (PEBAX), nylon, composites, or any combination of the above, or the like, or metals such as stainless steel, nitinol, or the like.

In operation, the toroidal balloon 50 is advanced through the vasculature with the catheter 40. The toroidal balloon 50 is inflated with a fluid, such as contrast saline, by hand injection with a syringe or other pressure source. After the toroidal balloon 50 has been used for the desired task, the fluid is withdrawn from the toroidal balloon 50 by pulling back on the syringe to deflate and collapse the toroidal balloon 50. The toroidal balloon 50 can then be withdrawn from the vasculature with the catheter 40. In one embodiment, the toroidal balloon system can include an integrated sheath to receive the toroidal balloon 50 when collapsed. The integrated sheath can keep the collapsed balloon from catching on parts of the anatomy during retraction.

The path through the vasculature by the balloon catheter is determined by the particular task for which the toroidal balloon 50 is to be used. For example, when the task is molding a stent graft for fit and seal, the approach can be through the femoral artery or the carotid artery. Multiple toroidal balloons can be used simultaneously. For example, a toroidal balloon with an embolic net can be placed through entry brachially to catch any emboli loosed by the procedure and a toroidal balloon for stent graft molding can be placed through entry in the groin. Different approaches can also be used to avoid conflicting catheter placement. For example, a toroidal balloon for stent graft molding can be placed through entry brachially and the stent graft delivery and deployment can be made through entry in the groin.

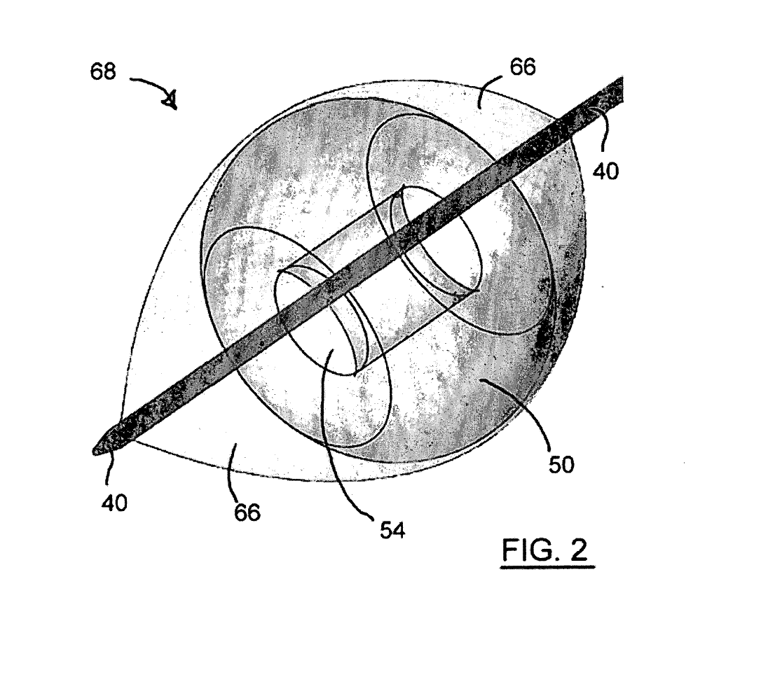

FIG. 2 is a schematic perspective view of the toroidal balloon system of FIGS. 1A-1C with an embolic net. In this embodiment, the embolic net 66 is disposed over the toroidal balloon 50 and attached to the catheter 40. The toroidal balloon system 68 can be used to perform the combination of stent graft molding and emboli catching, or can be disposed in a vessel for emboli catching alone. The inflation pressure in the toroidal balloon 50 assures that the embolic net 66 is effective since the toroidal balloon 50 provides a good seal with the vessel wall. The embolic net 66 can be attached to the catheter 40 with an adhesive, heat bonding, crimpable fittings, or the like.

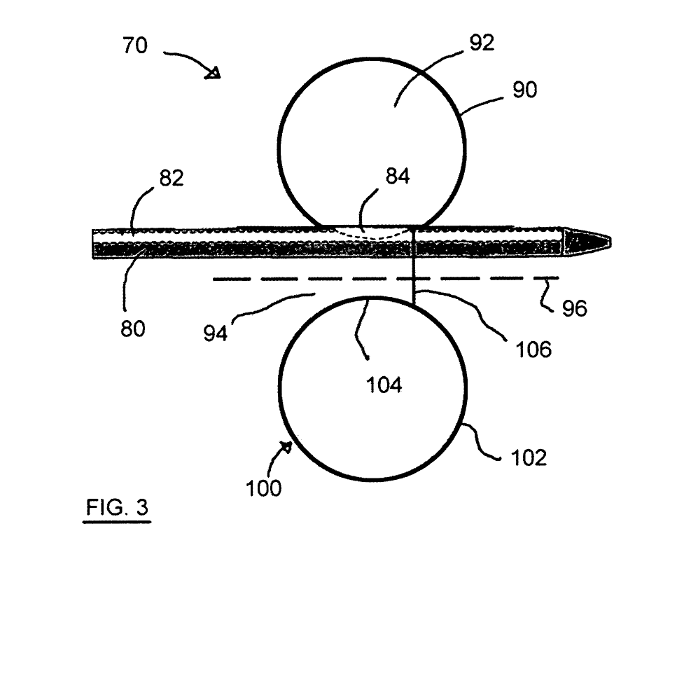

FIG. 3 is a schematic cross section view of another embodiment of a toroidal balloon system. In this embodiment, the planar closed curve of the torroidal balloon is generally circular. The toroidal balloon system is shown with the toroidal balloon inflated. The toroidal balloon system 70 includes a catheter 80 and a toroidal balloon 90 attached to the catheter 80. The catheter 80 defines an inflation lumen 82 and has an inflation port 84 in communication with the inflation lumen 82. The inflation port 84 can be skived into the inflation lumen 82 of the catheter 80. The toroidal balloon 90 defines a balloon lumen 92 in communication with the inflation port 84. When the toroidal balloon 90 is inflated, the toroidal balloon 90 defines a central lumen 94 for fluid flow through the vessel in which the toroidal balloon 90 is deployed. The toroidal balloon 90 has a balloon axis 96 through the central lumen 94. In this embodiment, the planar closed curve 100 is generally circular, with the portion 102 of the closed curve 100 away from the balloon axis 96 being generally semicircular and the portion 104 of the closed curve 100 near the balloon axis 96 being generally semicircular as well. An optional embolic net 106 is disposed across the central lumen 94.

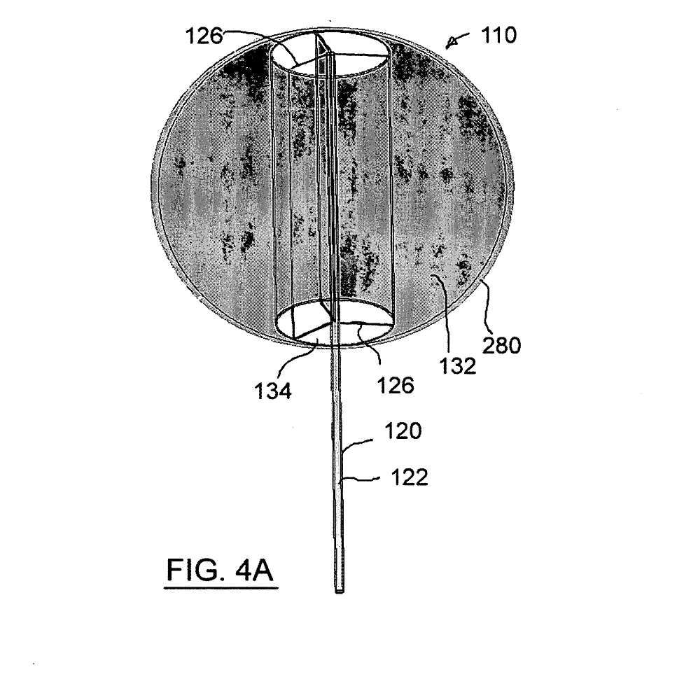

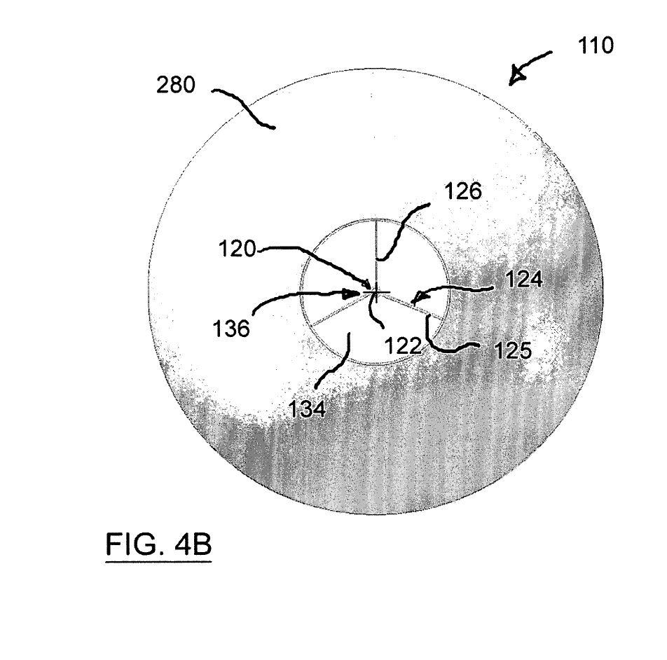

FIGS. 4A & 4B are schematic side and end views, respectively, of another embodiment of a toroidal balloon system. In this embodiment, the planar closed curve of the torroidal balloon is generally D-shaped and the catheter is attached to the torroidal balloon with ribs in the central lumen. The toroidal balloon system 110 includes a catheter 120 and a toroidal balloon 280 attached to the catheter 120 with ribs 126. The ribs 126 can also be inflation ribs. The catheter 120 defines an inflation lumen 122 and has one or more inflation ribs 124 having an inflation rib lumen 125 in communication with the inflation lumen 122. The catheter 120 can define additional lumens, such as a guidewire lumen, as desired for a particular application. The toroidal balloon 280 defines a balloon lumen 132 in communication with the inflation rib lumen 125. When the toroidal balloon 280 is inflated, the toroidal balloon 280 defines a central lumen 134 for fluid flow through the vessel in which the toroidal balloon 280 is deployed. The toroidal balloon 280 has a balloon axis 136 through the central lumen 134. The ribs 126 can position the catheter 120 on or off the balloon axis 136 as desired for a particular application.

The ribs 126 and/or the inflation rib 124 can be made of the same materials as the catheter 120 and/or the toroidal balloon 280. In one embodiment, the ribs 126 and/or the inflation rib 124 are rigid or semi-rigid. To provide a low profile for passage through the vasculature, the ribs 126 and/or the inflation rib 124 can be rolled and/or pinwheeled about the catheter 120.

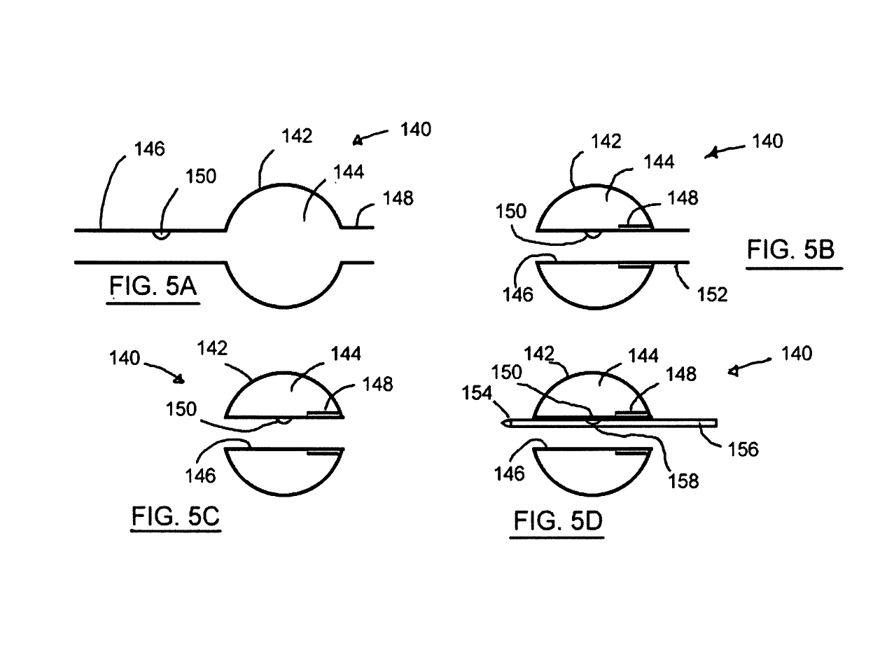

FIGS. 5A-5D are schematic progressive cross sectional views of a method of manufacturing a toroidal balloon system. Referring to FIG. 5A, a toroidal balloon blank 140 is provided. The toroidal balloon blank 140 includes a balloon body 142 defining a balloon lumen 144, a first leg 146 attached to the balloon body 142, and a second leg 148 attached to the balloon body 142 opposite the first leg 146. In this embodiment, the first leg 146 includes a balloon inflation port 150, which can be molded or cut into the first leg 146. Referring to FIG. 5B, the first leg 146 is inverted and folded through the balloon lumen 144 and into the second leg 148. The second leg 148 is inverted and folded into the balloon lumen 144 about the first leg 146. Referring to FIG. 5C, the first leg 146 is glued to the second leg 148 at the joint 151 where the first leg 146 meets the second leg 148. The excess 152 of the first leg 146 can be trimmed flush with the balloon body 142. Referring to FIG. 5D, a catheter 154 defining a catheter lumen 156 and having a catheter inflation port 158 is provided. The balloon inflation port 150 is aligned with the catheter inflation port 158 so the catheter lumen 156 communicates with the balloon lumen 144, and the catheter 154 is sealed to the toroidal balloon blank 140 where the catheter 154 and the toroidal balloon blank 140 meet. Adhesive sealant can be applied along both sides of the catheter 154 for the full length of the first leg 146 to ensure a seal.

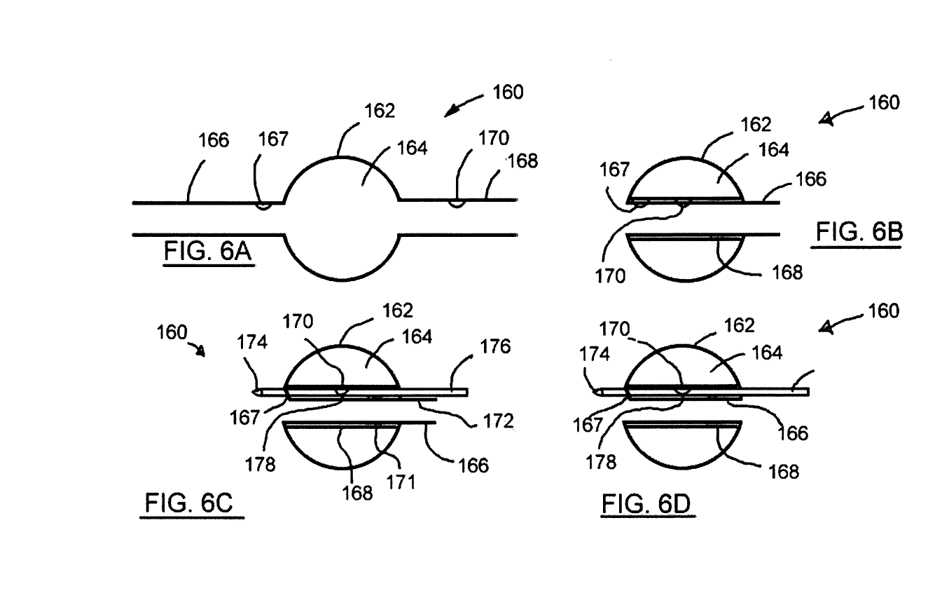

FIGS. 6A-6D are schematic progressive cross section views of another method of manufacturing a toroidal balloon system. Referring to FIG. 6A, a toroidal balloon blank 160 is provided. The toroidal balloon blank 160 includes a balloon body 162 defining a balloon lumen 164, a first leg 166 attached to the balloon body 162, and a second leg 168 attached to the balloon body 162 opposite the first leg 166. In this embodiment, the first leg 166 defines a catheter exit hole 167, which can be molded or cut into the first leg 166, and the second leg 168 includes a balloon inflation port 170, which can be molded or cut into the second leg 168. Referring to FIG. 6B, the first leg 166 is inverted and folded through the balloon lumen 164 and into the second leg 168. The second leg 168 is inverted and folded into the balloon lumen 164 about the first leg 166. Referring to FIG. 6C, a catheter 174 defining a catheter lumen 176 and having a catheter inflation port 178 is provided. The catheter 174 is inserted between the first leg 166 and the second leg 168, with the tip of the catheter 174 exiting the catheter exit hole 167. The balloon inflation port 170 is aligned with the catheter inflation port 178 so the catheter lumen 176 communicates with the balloon lumen 164. Referring to FIG. 6D, the first leg 166 is glued to the second leg 168 at the joint 171 where the first leg 166 meets the second leg 168. Adhesive sealant can be applied along the length of the catheter 174 for the full length of the first leg 166 to ensure a seal, so that the catheter 174 is sealed to the toroidal balloon blank 160 where the catheter 174 and the toroidal balloon blank 160 meet. The excess 172 of the first leg 166 can be trimmed flush with the balloon body 162.

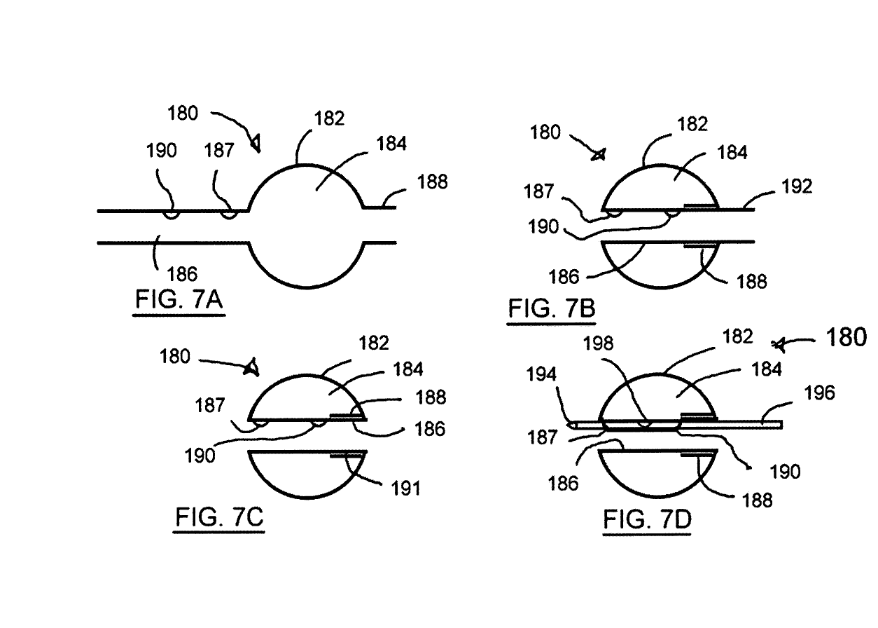

FIGS. 7A-7D are schematic progressive cross section views of another method of manufacturing a toroidal balloon system. Referring to FIG. 7A, a toroidal balloon blank 180 is provided. The toroidal balloon blank 180 includes a balloon body 182 defining a balloon lumen 184, a first leg 186 attached to the balloon body 182, and a second leg 188 attached to the balloon body 182 opposite the first leg 186. In this embodiment, the first leg 186 defines a catheter entrance hole 190 and a catheter exit hole 187, which can be molded or cut into the first leg 186. Referring to FIG. 7B, the first leg 186 is inverted and folded through the balloon lumen 184 and into the second leg 188. The second leg 188 is inverted and folded into the balloon lumen 184 about the first leg 186. Referring to FIG. 7C, the first leg 186 is glued to the second leg 188 at the joint 191 where the first leg 186 meets the second leg 188. The excess 192 of the first leg 186 can be trimmed flush with the balloon body 182. Referring to FIG. 7D, a catheter 194 defining a catheter lumen 196 and having a catheter inflation port 198 is provided. The tip of the catheter 194 is inserted into the catheter entrance hole 190 and out the catheter exit hole 187. The catheter inflation port 198 is within the balloon lumen 184, so the catheter lumen 196 communicates with the balloon lumen 184. The catheter 194 is sealed to the toroidal balloon blank 180 where the catheter 194 and the toroidal balloon blank 180 meet by applying adhesive sealant around the catheter entrance hole 190 and out the catheter exit hole 187.

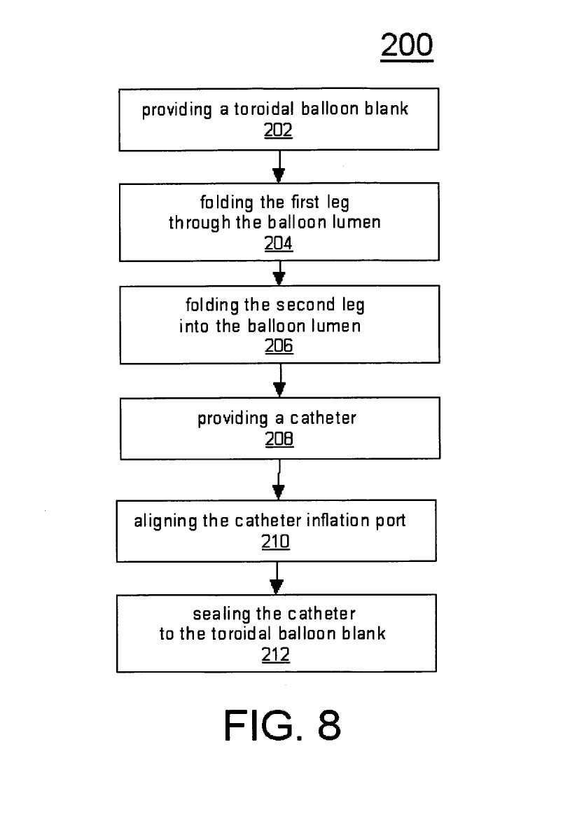

FIG. 8 is a flowchart of a method of manufacturing a toroidal balloon system. The method 200 includes providing a toroidal balloon blank (202) having a balloon body defining a balloon lumen, a first leg attached to the balloon body, and a second leg attached to the balloon body opposite the first leg; folding the first leg through the balloon lumen (204) and into the second leg; folding the second leg into the balloon lumen (206) about the first leg; providing a catheter (208) defining a catheter lumen and having an catheter inflation port; aligning the catheter inflation port (210) so the catheter lumen communicates with the balloon lumen; and sealing the catheter to the toroidal balloon blank (212).

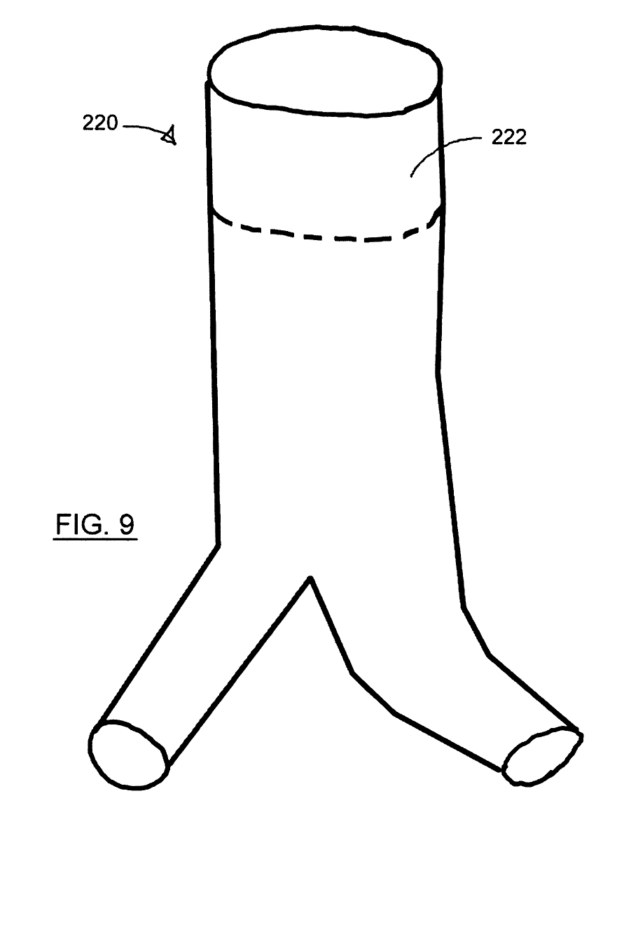

FIGS. 9-14 describe a system and method of deploying a graft with a double toroidal balloon system. The graft and the vessel wall are stained with Rose Bengal, the stained portions placed in close proximity to each other, and the stained portions exposed to light to bond the graft to the vessel wall. The light is maintained on the stained portions long enough to deliver sufficient energy to crosslink the stain.

FIG. 9 is a schematic side view of a graft. The graft has at least one stained graft portion to allow photofixation with a stained vessel wall portion of a vessel. The graft 220 is a generally tubular device having a stained graft portion 222. The stained graft portion 222 is stained with Rose Bengal (4,5,6,7-tetrachloro-2′,4′,5′,7′-tetraiodofluorescein), which binds to the collagen in the graft 220.

The graft 220 can be any tubular graft including collagenous material. The graft 220 can be a simple tube or can be bifurcated, with extensions of even or uneven length. The size and configuration of graft 220 are chosen to match the size and configuration of the vessel to be treated. The material of which the graft 220 is constructed can be any collagenous material. For example, the graft 220 can be made of a biologic material such as human amniotic membrane, other vessels, or other human or animal biologic material. Use of biologic material promotes ingrowth of the vessel into the graft 220. In another example, the graft 220 can be made of a polymer with a patch of biologic material in the stained graft portion 222. The graft 220 can include a support, such as a small wire diameter ring of nitinol or stainless steel, in the stained graft portion 222 to help retain the graft 220 on the light delivery balloon during delivery. In one embodiment, the graft 220 can be made of thin material allowing the graft 220 to be rolled to a small diameter, such as less than 14 French or less than 10 French, over a light delivery balloon. A small diameter allows access to smaller vessels in the vasculature.

The stained graft portion 222 of the graft 220 is stained with Rose Bengal (4,5,6,7-tetrachloro-2′,4′,5′,7′-tetraiodofluorescein). Rose Bengal is not an adhesive, but binds to collagen in tissue without adding volume and does not embolize or create embolic particles. When two stained portions are approximate and exposed to light, such as 530 nm laser light, 514 nm laser light, or other laser light effective to cross link the Rose Bengal, an adhesive connection/covalent bond is formed between the two stained portions through cross linking of the Rose Bengal in the two stained portions. The light can be applied through the tissue of the graft 220. The graft 220 can include one or more stained portions, such as a stained portion at the distal end and a stained portion at the proximal end to fix both ends of the graft to the vessel.

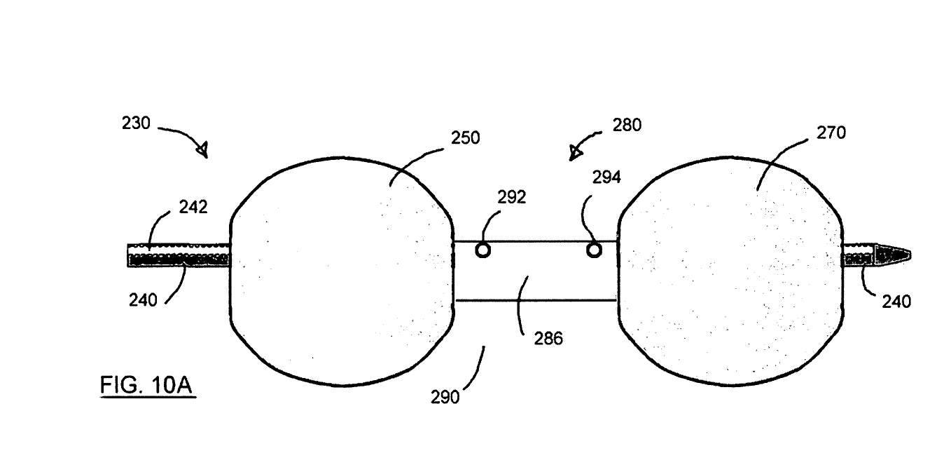

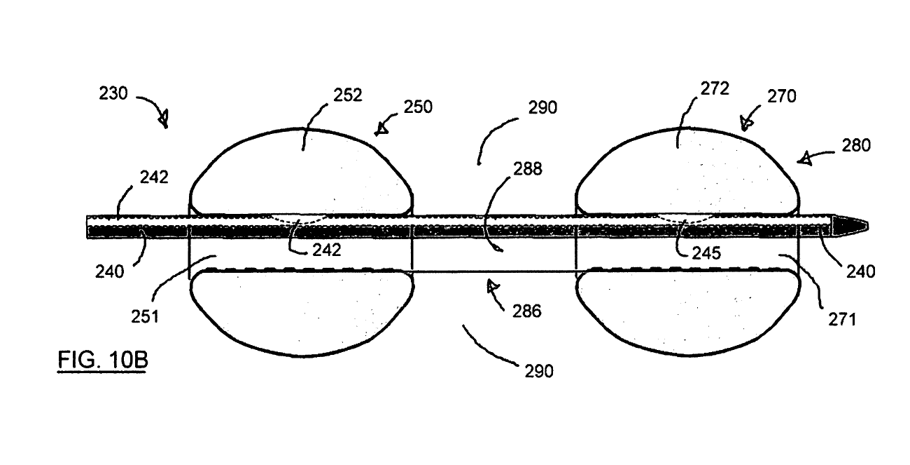

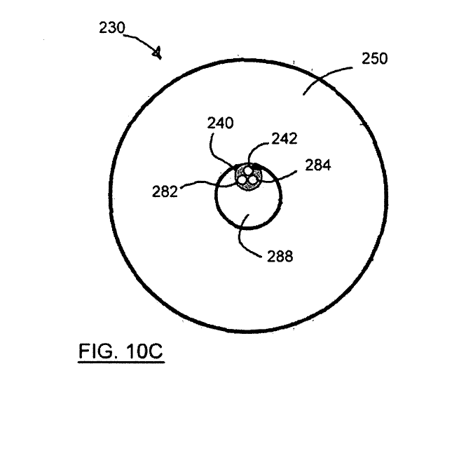

FIGS. 10A-10C are schematic side, cross section, and end views, respectively, of a double toroidal balloon system. In this embodiment, a double balloon isolates at least a portion of a vessel. The vessel portion is stained to allow attachment by photofixation of a graft with a stained graft portion. Each of the toroidal balloons can be similar to the toroidal balloons described in conjunction with the FIGS. 1A-1C, 3, and 4A & 4B above.

Referring to FIGS. 10A-10C, the double toroidal balloon system 230 includes a catheter 240 and a double balloon 280. The catheter 240 defines an inflation lumen 242, a supply lumen 282, and a return lumen 284 through the length of the catheter 240. The double balloon 280 includes balloons 250, 270 attached to the catheter 240, each of the balloons 250, 270 having a central lumen 251, 271 and having a balloon lumen 252, 272 communicating with the inflation lumen 242 through an inflation port 244, 245. A perfusion body 286 including a perfusion opening 288 connects the central lumen 251, 271 of the balloons 250, 270, so that blood can flow through the perfusion body 286 of the double toroidal balloon system 230 when it is deployed in a vessel. Adjoining exterior surfaces of the balloon 250, the perfusion body 286, and the balloon 270 define an isolation region 290 when the balloons 250, 270 are inflated in a vessel. The isolation region 290 is in communication with the supply lumen 282 through supply port 292 and the return lumen 284 through return port 294, so fluid can be supplied and returned from the isolation region 290. In another embodiment, the balloons 250, 270 are each connected to an independent inflation lumen so the balloons can be inflated and deflated independently of each other. Those skilled in the art will appreciate that the balloons 250, 270 can be any balloons capable of establishing the isolation zone 290 and are not limited to toroidal balloons.

The double balloon 280 is sized to fit over the region of the vessel to be stained and the inflation lumen 242 inflates the balloons 250, 270 to maintain the double balloon 280 over the region. The balloons 250, 270 are separated by a distance along the catheter 240, the distance being selected to accommodate the region to be stained in the isolation region. In operation, the distal end of the double toroidal balloon system 230 is delivered to region to be stained through a delivery catheter in a rolled and/or folded configuration. The double balloon 280 unfurls on exiting the delivery catheter and is inflated to the illustrated configuration. The double balloon 280 is deflated and retracted into the delivery catheter after the vessel has been stained. The diameter of the perfusion body 286 can be selected to provide a desired volume in the isolation region 290.

The perfusion body 286 can be made of any flexible biocompatible material normally used for catheter balloons. For example, the perfusion body 286 can be made of polymers such as polyethylene, polyethylene terephalate (PET), nylon, polyurethane, polyether block amide (PEBAX), polyetheretherketone (PEEK), or the like.

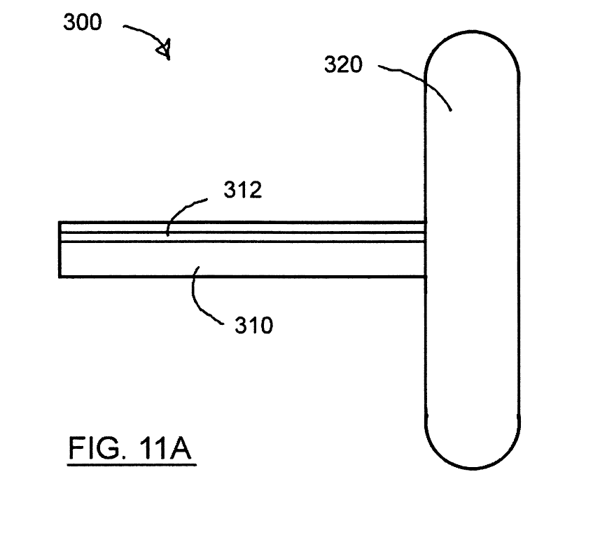

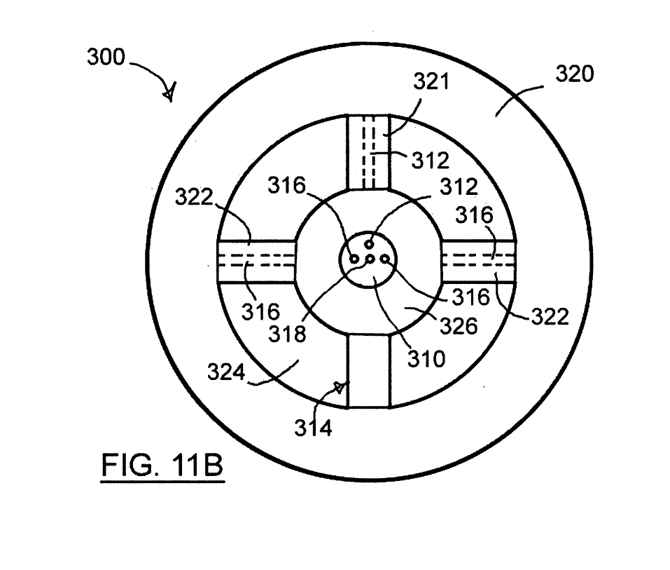

FIGS. 11A & 11B are schematic side and end views, respectively, of a light delivery balloon system. The light delivery balloon system delivers the graft to the stained portion of the vessel wall, aligns and holds the stained portion of the vessel with the stained graft portion of the graft, and provides a path for exposing the stained portions with light to bond the graft to the vessel wall. In this embodiment, the planar closed curve of the torroidal balloon is generally circular and the catheter is attached to the torroidal balloon with ribs in the central lumen.

The light delivery balloon system 300 includes a catheter 310 and a toroidal balloon 320 attached to the catheter 310 with hub 326 and ribs 314. The toroidal balloon 320 can be transparent or translucent to allow light to pass through the balloon to the graft. The catheter 310 defines an inflation lumen 312, and one or more light catheter lumens 316. The catheter 310 can define additional lumens, such as a guidewire lumen 318, as desired for a particular application. The ribs 314 can be solely structural, can be inflation ribs 321 continuing the inflation lumen 312, or can be light ribs 322 continuing the light catheter lumens 316. The number of light ribs 322 can be selected so light can be applied around the full circumference of the toroidal balloon 320. The hub 326 provides a rounded transition in the light catheter lumen 316 between the catheter 310 and the light rib 322 so that a light catheter moving distally in the light catheter lumen 316 can change direction. The toroidal balloon 320 defines a balloon lumen in communication with the inflation lumen 312. When the toroidal balloon 320 is inflated, the toroidal balloon 320 defines a central lumen 324 for fluid flow through the vessel in which the toroidal balloon 320 is deployed. The light delivery balloon system 300 can include an additional toroidal or conventional balloon proximal the toroidal balloon 320 on the catheter 310 to assist in supporting the graft during delivery and deployment. Those skilled in the art will appreciate that the toroidal balloon 320 can be any balloon capable of delivering and illuminating the graft and is not limited to a toroidal balloon.

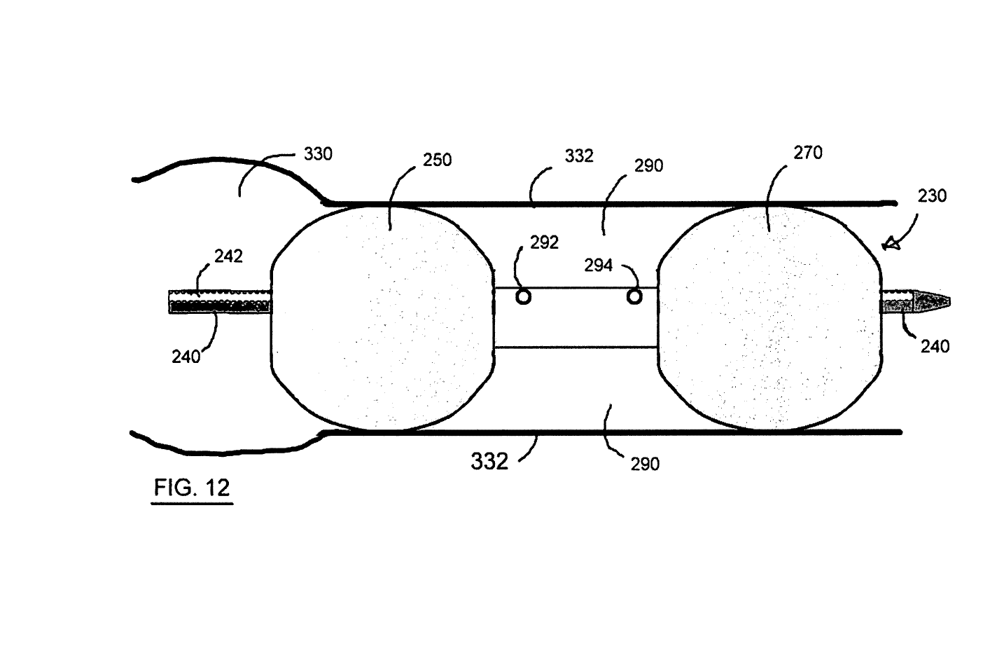

FIG. 12 is a schematic side view of a double toroidal balloon system as described in conjunction with FIGS. 10A-10C deployed in a vessel. Referring to FIG. 12, the double toroidal balloon system 230 has been deployed in a vessel proximal of an aneurysm 330 to stain vessel wall portion 332. The distal end of the double toroidal balloon system 230 has been delivered through a delivery catheter with the double balloon 280 in a rolled and/or folded configuration. The double balloon 280 has unfurled on exiting the delivery catheter and has been inflated to the illustrated configuration. To stain vessel wall portion 332, the isolation zone 290 and the vessel wall portion 332 can be rinsed by supplying a rinse fluid, such as a saline solution, from the supply port 292 into the isolation zone 290 and removing the rinse fluid from the isolation zone 290 through the return port 294. After the rinse fluid, a Rose Bengal stain solution is injected into the isolation zone 290 from the supply port 292. The Rose Bengal stain solution can be maintained in the isolation zone 290 for the time required for the Rose Bengal stain to stain the tissue of the vessel wall portion 332. The Rose Bengal stain solution can be flushed from the isolation zone 290 through the return port 294 or maintained in the isolation zone 290 for release into the vasculature with the deflation of the double balloon 280. The double balloon 280 is deflated and retracted into the delivery catheter. Alternatively, the double balloon 280 can be advanced or retracted and the staining repeated for another vessel wall portion. The vessel wall portion 332 has now been stained and is prepared for attachment of a graft.

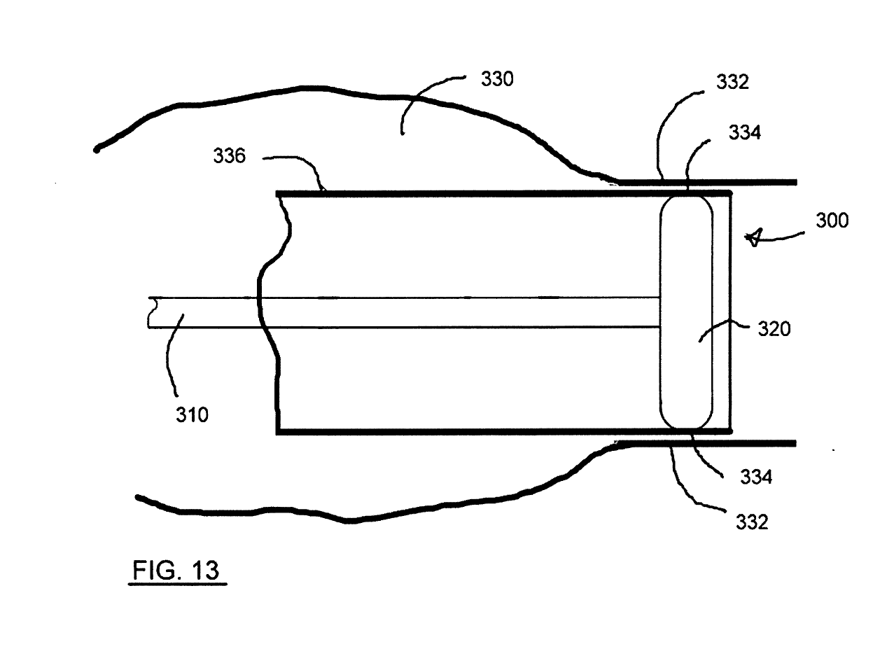

FIG. 13 is a schematic side view of a light delivery balloon system as described in conjunction with FIGS. 11A & 11B deployed to fix a graft 336 in a vessel. Referring to FIG. 13, the light delivery balloon system 300 has been deployed in a vessel proximal of an aneurysm 330 to bond a graft 336 to the vessel wall portion 332. The graft 336 is shown as transparent for clarity of illustration. The exterior stained graft portion 334 of the graft 336 is held in close proximity to the stained interior vessel wall portion 332 with the toroidal balloon 320. The distal end of the light delivery balloon system 300 has been delivered through a delivery catheter with the graft 336 and the toroidal balloon 320 in a rolled and/or folded configuration. The delivery catheter can be the delivery catheter used on staining the vessel wall portion 332 with the double toroidal balloon system. The graft 336 and the toroidal balloon 320 have unfurled on exiting the delivery catheter and the toroidal balloon 320 has been inflated to the illustrated configuration. A light catheter with a light source on the distal end is advanced through the catheter to provide light for attaching the stained graft portion 334 of the graft 336 to the stained vessel wall portion 332. In one embodiment, the light catheter is a fiberoptic cable with a laser source external to the patient. In another embodiment, the light source is a laser source or a light emitting diode (LED) source on the distal end of the light catheter and the light catheter provides an electrical path to power the laser or LED source. The light from the light source shines through the toroidal balloon 320 and the stained graft portion 334 of the graft 336, and crosslinks the Rose Bengal stain in the stained graft portion 334 and the vessel wall portion 332.

The light catheter can provide light around the circumference of the graft 336 so the graft 336 is sealed to the vessel wall portion 332 around the whole circumference. The light is maintained on the stained portions long enough to deliver sufficient energy to crosslink the stain. In one embodiment, the light delivery balloon system 300 includes a light catheter lumen the length of the catheter in communication with several radial light ribs at the hub of the toroidal balloon 320. The light catheter is steerable and is inserted in the light catheter lumen in one light rib, maintained in that light rib long enough for the light to the attach the graft to the vessel at that point, retracted until the distal tip is clear of that light rib, and steered to another light catheter lumen in another light rib. In another embodiment, the light delivery balloon system 300 includes a number of light catheter lumens the length of the catheter, each of the light catheter lumens being in communication with one radial light rib. The light catheter can be inserted in the light catheter lumens sequentially to expose the points on the circumference one at a time or multiple light catheters can be used simultaneously to expose the points on the circumference simultaneously. In yet another embodiment, the light delivery balloon system 300 includes a single light catheter lumen the length of the catheter, with the light catheter lumen in communication with a single radial light rib. The light catheter can be inserted in the light catheter lumen and the light delivery balloon rotated after each point is exposed until the whole circumference of the graft is sealed to the vessel wall.

After the graft is sealed to the vessel, the toroidal balloon 320 is deflated and retracted into the delivery catheter. Alternatively, the toroidal balloon 320 can be partially retracted and the sealing repeated for another stained graft portion of the graft 336 to another vessel wall portion, such as the other end of the graft 336 to the vessel distal the aneurysm 330.

The graft 336 can be held on the toroidal balloon 320 during delivery and deployment by folding the graft 336 into the toroidal balloon 320. In one embodiment, the graft 336 can be affixed to the toroidal balloon 320 with a light tack or soluble adhesive. In another embodiment, the graft 336 can be retained on the toroidal balloon 320 with a support, such as a small wire diameter ring of nitinol or stainless steel. In yet another embodiment, the graft 336 can be retained on the toroidal balloon 320 with a tether. In one example, the tether can sew the toroidal balloon 320 to the graft 336 and the tether can be clipped with a cutting catheter having a cutter on the distal portion after the graft has been fixed to the vessel. In another example, the tether can loop through the toroidal balloon 320 and the graft 336 so that both ends of the tether remain outside the patient, and the tether can be retracted by pulling one of the ends after the graft has been fixed to the vessel. The light delivery balloon system 300 can optionally include an additional toroidal or conventional balloon proximal the toroidal balloon 320 on the catheter 310 to assist in supporting the graft during delivery and deployment.

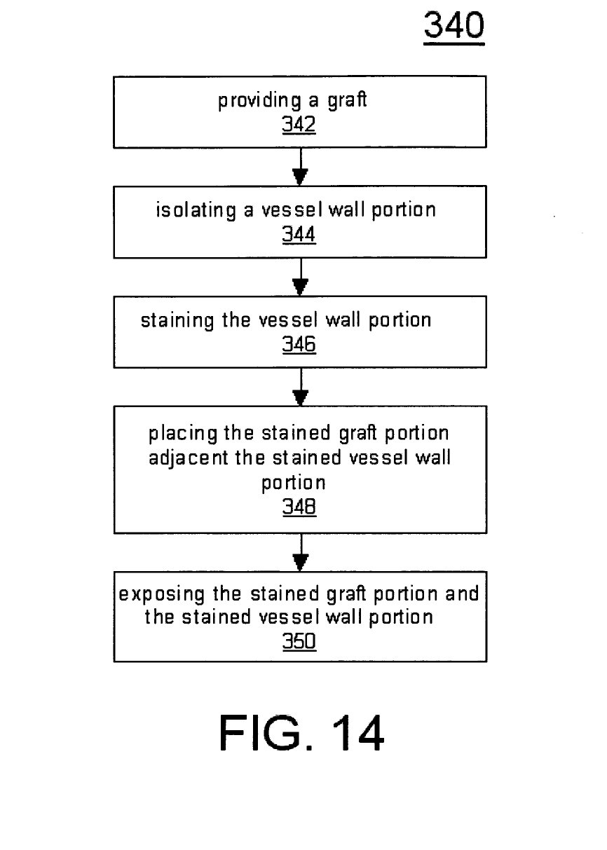

FIG. 14 is a flowchart of the steps of a method of deploying a graft in a vessel. The method 340 includes the steps of providing a graft (342) having a graft portion stained with Rose Bengal; isolating a vessel wall portion (344) from blood flow through the vessel without blocking the blood flow through the vessel; staining the vessel wall portion (346) with Rose Bengal; placing the stained graft portion adjacent the stained vessel wall portion (348); and exposing the stained graft portion and the stained vessel wall portion (350) with light to bond the stained graft portion and the stained vessel wall portion.

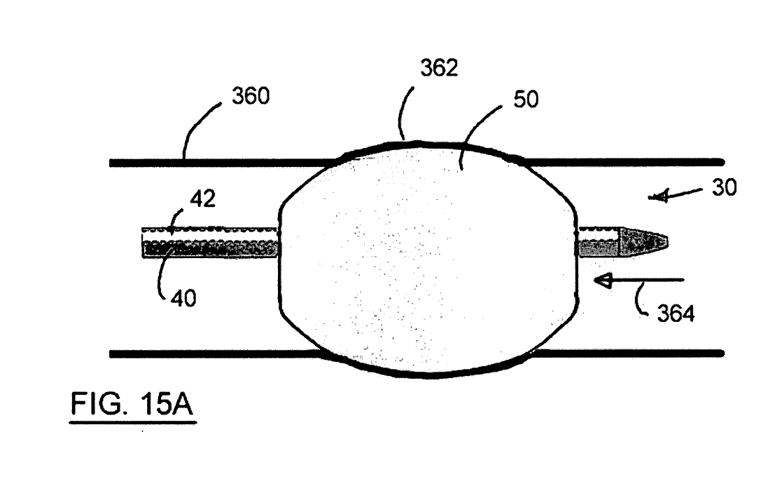

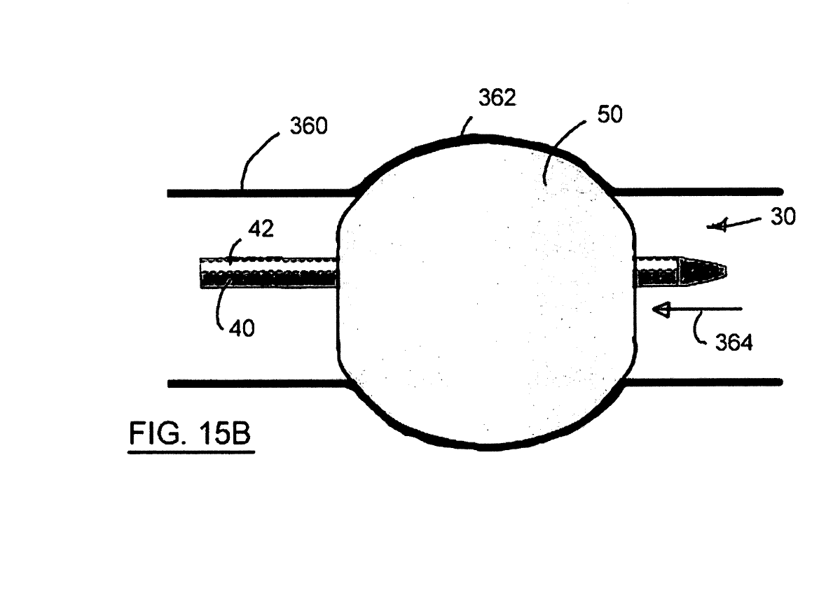

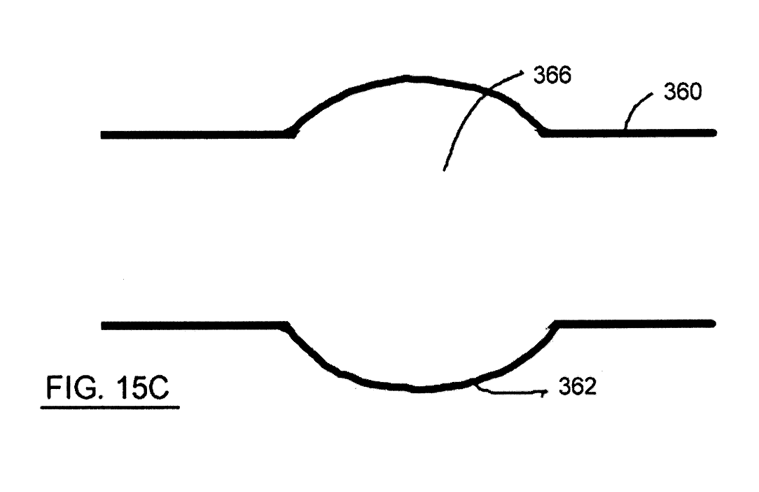

FIGS. 15A-15C are schematic progressive side views of developing an aneurysm with a toroidal balloon system. In this example, the toroidal balloon system is the toroidal balloon system described in conjunction with FIGS. 1A-1C.

Referring to FIG. 15A, the toroidal balloon 50 of the toroidal balloon system 30 is advanced to a target site 362 in a vessel 360. The toroidal balloon 50 is oversized for the vessel 360 and non-compliant to produce the bulge at the target site 362. The toroidal balloon 50 is inflated to a diameter greater than the initial vessel diameter at the target site 362. The toroidal balloon 50 is held at the target site 362 by friction and the radial force of the toroidal balloon 50 on the vessel 360. In the vasculature of quadrupeds, the aorta is horizontal so the gravitational loading also helps keep the toroidal balloon 50 in place. Blood flow 364 as indicated by the arrow is maintained through the vessel 360 by the passage of blood through the central lumen of the toroidal balloon 50. The toroidal balloon 50 is retained in the vessel 360 until the vessel diameter at the target site is fixed at the greater diameter of the toroidal balloon 50. Referring to FIG. 15B, the toroidal balloon 50 is inflated further to a diameter greater than the fixed greater vessel diameter at the target site 362 to increase the vessel diameter further. Referring to FIG. 15C, the toroidal balloon 50 is removed and the vessel 360 includes an aneurysm 366 at the target site 362.

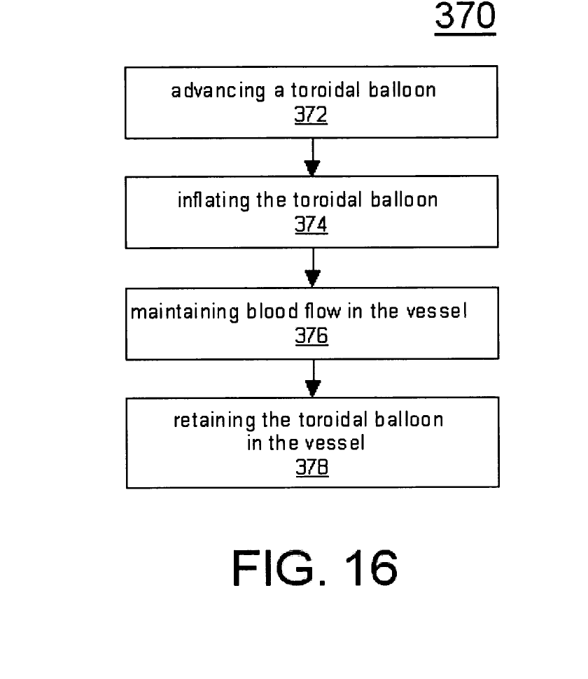

FIG. 16 is a flowchart of the steps of a method of developing an aneurysm in a vessel. The method 370 includes the steps of advancing a toroidal balloon (372) having a central lumen to a target site in the vessel; inflating the toroidal balloon (374) to a diameter greater than a vessel diameter at the target site; maintaining blood flow in the vessel (376) through the central lumen; and retaining the toroidal balloon in the vessel (378) until the vessel diameter at the desired point is fixed at the greater diameter of the toroidal balloon. The steps of inflating the toroidal balloon and retaining the toroidal balloon in the vessel can be repeated until the aneurysm reaches a desired diameter. The toroidal balloon can be shifted axially and inflated at different target sites in the vessel to produce an aneurysm having a desired axial profile.

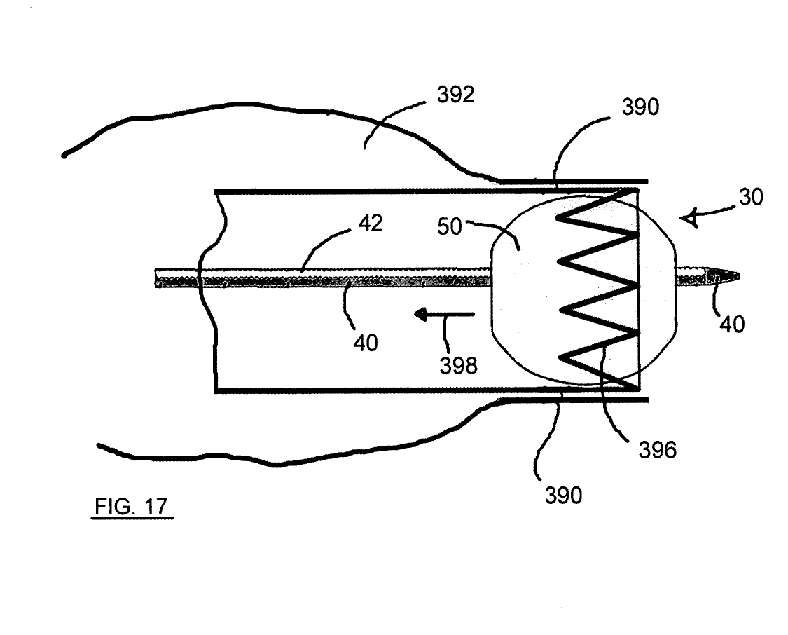

FIG. 17 is a schematic side view of a toroidal balloon system as described in conjunction with FIGS. 1A-1C deployed in a vessel with a stent graft. Referring to FIG. 17, a stent graft has been deployed in a vessel 390 across an aneurysm 392. The stent graft is shown as transparent for clarity of illustration. The stent graft has at least one stent 396 and a central lumen allowing blood flow across the toroidal balloon 50 when inflated. The distal end of the toroidal balloon system 30 has been delivered through a delivery catheter with the toroidal balloon 50 in a rolled and/or folded configuration. The delivery catheter can be the delivery catheter used to deliver the stent graft. The toroidal balloon 320 has unfurled on exiting the delivery catheter and has been inflated to the illustrated configuration with the toroidal balloon 50 inside the stent 396. The clinician inflates the toroidal balloon 50 to the diameter required to fit the stent 396 to the vessel 390. Blood flow as indicated by arrow 398 is maintained in the vessel 390 through the central lumen of the toroidal balloon 50, so the clinician is free to take time in making adjustments. The clinician can image the stent graft and vessel to determine the adjustments required and the final fit. The toroidal balloon 50 can be adjusted, inflated, and deflated repeatedly until the desired fit is achieved. After the stent 396 is fit to the vessel, the toroidal balloon 50 can be deflated and retracted into the delivery catheter. Alternatively, the toroidal balloon 50 can be partially retracted and the steps repeated for fitting another portion of the stent graft to the vessel 390, such as the other end of the stent graft to the vessel 390 distal to the aneurysm 392.

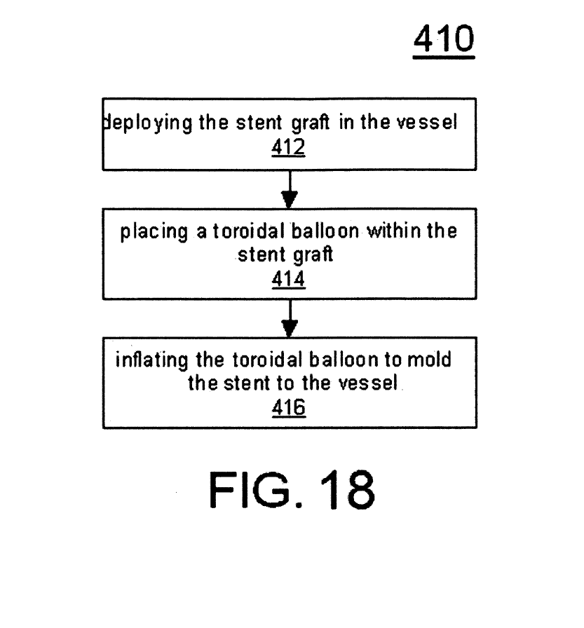

FIG. 18 is a flowchart of the steps of a method of molding a stent graft to a vessel. The method 410 includes the steps of deploying the stent graft in the vessel (412), the stent graft having at least one stent; placing a toroidal balloon within the stent graft (414) at the stent, the toroidal balloon having a central lumen; and inflating the toroidal balloon to fit the stent to the vessel (416) while maintaining blood flow in the vessel through the central lumen.

While specific embodiments according to the invention are disclosed herein, various changes and modifications can be made without departing from its spirit and scope.

1. A toroidal balloon system for use in a vessel comprising:
a catheter defining an inflation lumen and having an inflation port in communication with the inflation lumen; and
a toroidal balloon attached to the catheter, the toroidal balloon defining a balloon lumen in communication with the inflation port and a central lumen for fluid flow through the vessel when the toroidal balloon is inflated.

2. The system of claim 1 wherein the toroidal balloon is defined by rotation of a planar closed curve about a balloon axis and the planar closed curve is generally D-shaped.

3. The system of claim 1 wherein the toroidal balloon is defined by rotation of a planar closed curve about a balloon axis and the planar closed curve is generally circular.

4. The system of claim 1 wherein the toroidal balloon is attached to the catheter with ribs.

5. The system of claim 4 wherein the balloon lumen is in communication with the inflation port through at least one of the ribs.

6. The system of claim 1 further comprising an embolic net disposed across the central lumen.

7. The system of claim 6 wherein the embolic net is disposed around the toroidal balloon and attached to the catheter.

8. The system of claim 6 wherein the embolic net is disposed within the central lumen.

9. The system of claim 1 wherein the toroidal balloon has an inner wall defining the central lumen, the inner wall including an internal sheath.

10. The system of claim 9 wherein the internal sheath is a common biaxial braid.

11. A method of manufacturing a toroidal balloon system comprising:
providing a toroidal balloon blank having a balloon body defining a balloon lumen, a first leg attached to the balloon body, and a second leg attached to the balloon body opposite the first leg;
folding the first leg through the balloon lumen and into the second leg;
folding the second leg into the balloon lumen about the first leg;
providing a catheter defining a catheter lumen and having an catheter inflation port;
aligning the catheter inflation port so the catheter lumen communicates with the balloon lumen; and
sealing the catheter to the toroidal balloon blank.

12. The method of claim 11 further comprising trimming the first leg flush with the balloon body.

13. The method of claim 11 wherein the first leg defines a balloon inflation port and the aligning comprises aligning the catheter inflation port so the catheter lumen communicates with the balloon lumen through the balloon inflation port.

14. The method of claim 11 wherein the first leg defines a catheter exit hole and the second leg defines a balloon inflation port, and the aligning comprises positioning the catheter between the first leg and the second leg an through the catheter exit hole so the catheter inflation port aligns with the balloon inflation port.

15. The method of claim 11 wherein the first leg defines a catheter entrance hole and a catheter exit hole, and the aligning comprises positioning the catheter in the catheter entrance hole and out the catheter exit hole so the catheter inflation port communicates with the balloon lumen.

16. A method of molding a stent graft to a vessel comprising:
deploying the stent graft in the vessel, the stent graft having at least one stent;
placing a toroidal balloon within the stent graft at the stent, the toroidal balloon having a central lumen; and
inflating the toroidal balloon to fit the stent to the vessel while maintaining blood flow in the vessel through the central lumen.

17. A method of deploying a graft in a vessel comprising:
providing a graft having a graft portion stained with Rose Bengal;
isolating a vessel wall portion from blood flow through the vessel without blocking the blood flow through the vessel;
staining the vessel wall portion with Rose Bengal;
placing the stained graft portion adjacent the stained vessel wall portion; and
exposing the stained graft portion and the stained vessel wall portion with light to bond the stained graft portion and the stained vessel wall portion.

18. The method of claim 17 further comprising rinsing the vessel wall portion before the staining.

19. The method of claim 17 wherein the isolating comprises isolating the vessel wall portion from blood flow with a double balloon.

20. A method of developing an aneurysm in a vessel comprising:
advancing a toroidal balloon having a central lumen to a target site in the vessel;
inflating the toroidal balloon to a diameter greater than an initial vessel diameter at the target site;
maintaining blood flow in the vessel through the central lumen; and
retaining the toroidal balloon in the vessel until the vessel diameter at the target site is fixed at the greater diameter of the toroidal balloon.

21. The method of claim 20 further comprising inflating the toroidal balloon to a diameter greater than the vessel diameter at the target site after the retaining.

22. A system for graft deployment in a vessel comprising:
a graft having a stained graft portion;
a double toroidal balloon system comprising:
a first catheter defining a supply lumen and a return lumen; and
a double balloon attached to the first catheter, the double balloon having a first balloon connected to a second balloon with a perfusion body, the first balloon having a first central lumen, the second balloon having a second central lumen, the perfusion body having a perfusion opening connecting the first central lumen and the second central lumen;
wherein the supply lumen and the return lumen communicate to outside the perfusion body; and
a light delivery balloon system comprising:
a second catheter defining a light catheter lumen; and
a toroidal balloon attached to the second catheter;
wherein the double toroidal balloon system is operable to isolate a vessel wall portion of the vessel and deliver stain to the vessel wall portion through the supply lumen to generate a stained wall portion;
the light delivery balloon system is operable to align the stained graft portion with the stained wall portion; and
the light delivery balloon system is further operable to deliver a light catheter through the light catheter lumen to expose the aligned stained graft portion and the stained wall portion with light.
